(19) United States
(12) Patent Application Publication
(10) Pub. No.: US 2026/0360497 A1
(43) Pub. Date: Nov. 12, 2026

(54) APPARATUS, METHOD, AND PROGRAM FOR DETERMINING A DOSE OF AN ANTIBODY DRUG

(71) Applicant: CHUGAI SEIYAKU KABUSHIKI KAISHA, Tokyo (JP)

(72) Inventors: Yasunori KOMORI, Kanagawa (JP); Taichi AKAHOSHI, Kanagawa (JP); Tatsuhiko TACHIBANA, Kanagawa (JP); Koki KIBE, Kanagawa (JP); Kuniyasu KATO, Kanagawa (JP); Yoshie TAKAO, Kanagawa (JP)

(73) Assignee: CHUGAI SEIYAKU KABUSHIKI KAISHA, Tokyo (JP)

(21) Appl. No.: 19/663,314

(22) Filed: Apr. 30, 2026

(30) Foreign Application Priority Data
May 9, 2025 (JP) 2025-078707

(51) Int. Cl. G16H 20/10; G16B 20/00

(52) U.S. Cl. CPC G16H 20/10; G16B 20/00

(57) ABSTRACT

A system, method, and non-transitory computer-readable medium determine a dose of an antibody drug for administration to a subject. A memory stores a physiologically based pharmacokinetic (PBPK) model that includes a plurality of biological compartments and model parameters representing inflow, outflow, and elimination of a reference antibody in each compartment. The model parameters for elimination include an internalization rate at which an antigen-antibody complex is internalized into cells of an organ. Circuitry receives a candidate dose of a prediction target antibody, calculates a predicted plasma concentration time profile using the PBPK model, determines a dose for administration to the subject based on the predicted profile, and outputs the determined dose.

### CROSS-REFERENCE TO RELATED APPLICATION

This application claims priority to Japanese Patent Application No. 2025-078707, filed on May 9, 2025, the entire contents of which are incorporated herein by reference.

### TECHNICAL FIELD

The present disclosure relates to a prediction system, a prediction method, and a prediction program.

### BACKGROUND ART

Conventionally, techniques for predicting the concentration of an antibody in blood have been known. For example, in NPL 1, biological organs are represented as compartments and it is assumed that receptors are present in each compartment, and the parameters of a prediction model are optimized so that the predicted blood antibody concentration-time profile matches the observed concentration-time profile.

### CITATION LIST

### Non Patent Literature

NPL 1: Glassman P M, Balthasar J P. Physiologically-based pharmacokinetic modeling to predict the clinical pharmacokinetics of monoclonal antibodies. J Pharmacokinet Pharmacodyn. 2016 August; 43(4):427-46. doi: 10.1007/s10928-016-9482-0. Epub 2016 Jul. 4. PMID: 27377311.

### SUMMARY

### Technical Problem

However, the model described in NPL 1 does not sufficiently represent the actual in vivo kinetics of the antibody.

A system of the present disclosure for determining a dose of an antibody for administration to a subject includes a memory and circuitry. The memory stores a physiologically based pharmacokinetic (PBPK) model. The PBPK model includes a plurality of biological compartments and, as model parameters, inflow of a reference antibody into each compartment, outflow of the reference antibody from each compartment, and elimination of the reference antibody in each compartment. The model parameters for elimination include an internalization rate at which an antigen-antibody complex is internalized into cells of an organ, the antigen-antibody complex being formed by binding between the reference antibody transferred from plasma to the organ and an antigen protein possessed by the organ. The PBPK model is constructed based on pharmacokinetic data of the reference antibody. The circuitry receives input information including a candidate dose of a prediction target antibody; calculates, using the PBPK model and the candidate dose, a predicted time profile of a plasma concentration of the prediction target antibody; determines, based on the predicted time profile, a dose of the prediction target antibody for administration to the subject; and outputs the determined dose.

### Advantageous Effect

According to the present disclosure a concentration-time profile of an antibody in blood can be predicted based on a prediction model that reflects in vivo kinetics while ensuring applicability.

### BRIEF DESCRIPTION OF DRAWINGS

FIG. 1 is a block diagram showing an overview of the configuration of a prediction system according to a first embodiment of the present disclosure.

FIG. 2 is a block diagram showing the configuration of a prediction apparatus included in the prediction system according to the first embodiment.

FIG. 3 is a block diagram showing the configuration of a prediction model used in the prediction system according to the first embodiment.

FIG. 4 is a block diagram showing the configuration of a model construction apparatus included in the prediction system according to the first embodiment.

FIG. 5 is graphs illustrating how the prediction system according to the first embodiment determines parameters by the model construction apparatus.

FIG. 6 is a block diagram showing an example of the hardware configuration of a prediction apparatus included in the prediction system according to the first embodiment.

FIG. 7 is a block diagram showing an example of the hardware configuration of a model construction apparatus included in the prediction system according to the first embodiment.

FIG. 8 is a flowchart illustrating an example of an operation of the model construction apparatus included in the prediction system according to the first embodiment.

FIG. 9 is a flowchart illustrating an example of an operation of the model construction apparatus included in the prediction system according to the first embodiment.

FIG. 10 is a flowchart illustrating an example of an operation of the prediction apparatus included in the prediction system according to the first embodiment.

FIG. 11 is a block diagram showing the configuration of a model construction apparatus included in a prediction system according to a second embodiment.

FIG. 12 is a schematic diagram to illustrate a correction unit of the prediction system according to the second embodiment.

FIG. 13 is a flowchart illustrating an example of description of an operation of the model construction apparatus included in the prediction system according to the second embodiment.

FIG. 14 is graphs to illustrate an effect of the prediction system according to the second embodiment.

### DESCRIPTION OF EMBODIMENTS

An object of the present disclosure is to predict the concentration-time profile of an antibody in blood based on a prediction model that reflects in vivo kinetics while ensuring applicability, and to provide a dose-determination tool based on such prediction. However, the model described in NPL 1 does not sufficiently represent the actual in vivo kinetics of an antibody.

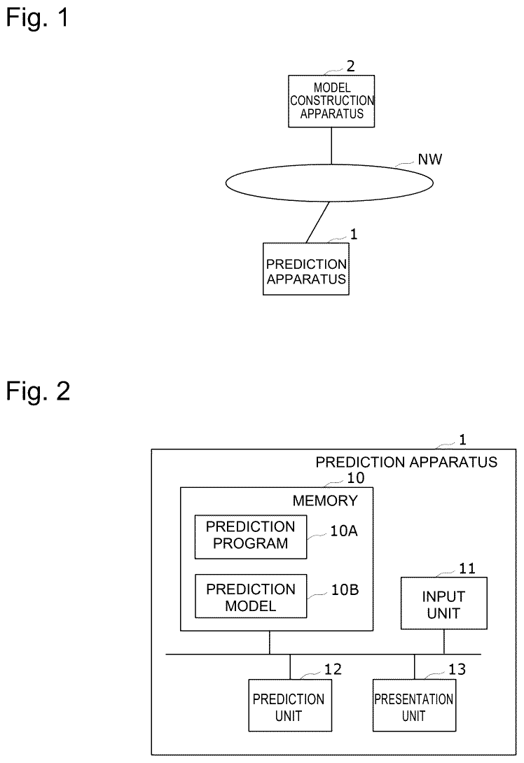

Hereinafter, embodiments of the present disclosure will be described in detail with reference to FIGS. 1 to 14.

### First Embodiment

First, an overview of a prediction system according to the first embodiment is described.

In the prediction system according to the first embodiment, a prediction apparatus 1 and a model construction apparatus 2 are connected via a network NW such as a LAN, a WAN, or the Internet. As one example, the prediction apparatus 1 acquires, via the network NW, a prediction model (physiologically based pharmacokinetic (PBPK) model) constructed by the model construction apparatus 2, and performs prediction processing locally. The model construction apparatus 2 is configured as a cloud server. The number of prediction apparatuses 1 included in the prediction system is not limited to 1, and a configuration with multiple prediction apparatuses 1 may be adopted.

The prediction system constructs a prediction model that reproduces a process in which an antibody of an intravenously administered antibody medicament is distributed, metabolized, and excreted in vivo on the basis of physiological and anatomical parameters, and predicts a time profile of the concentration of a prediction target antibody. In this embodiment, a model for predicting a time profile of an antibody concentration is constructed based on a physiologically based pharmacokinetic (PBPK) model that models a human body as a plurality of biological compartments. In the present disclosure, the antibody includes an antibody fragment if it can bind to an antigen, namely a binding target protein.

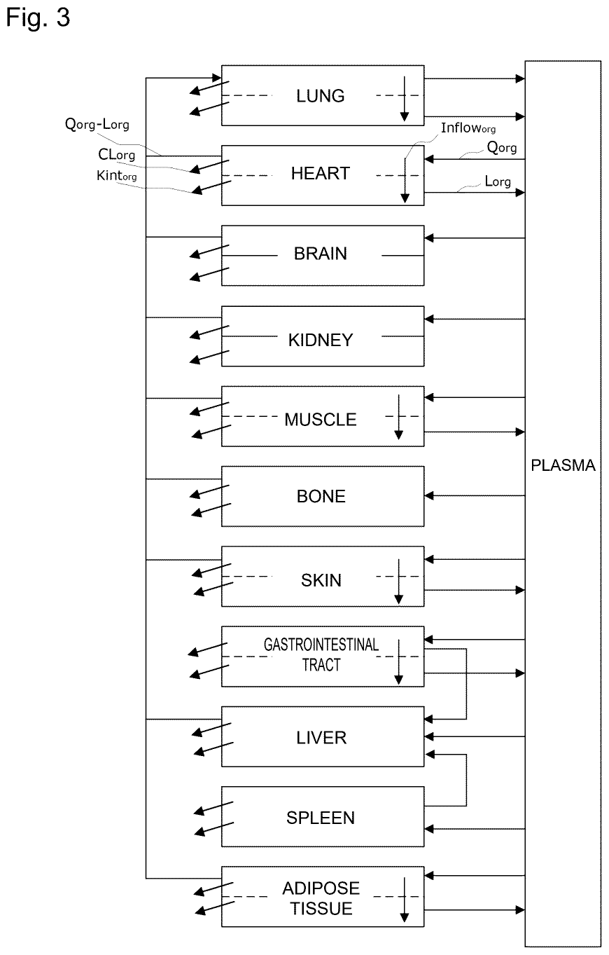

FIG. 3 is a schematic diagram illustrating a model constructed by the prediction system according to this embodiment. As shown in FIG. 3, the prediction model includes a plurality of organ compartments and a plasma compartment. Organs used in the prediction model are major organs selected in advance from among organs constituting the body. For example, organs whose weight is equal to or greater than a predetermined value relative to the total weight of organs are selected as organ compartments. The following describes a case where lung, heart, brain, kidney, muscle, bone, skin, gastrointestinal tract, liver, spleen, and adipose tissue are adopted.

In this prediction model, it is assumed that an intravenously administered antibody is administered into a plasma compartment. In practice, the antibody is administered intravenously; however, since the antibody rapidly distributes throughout systemic plasma such that any delay may be neglected in calculation, the antibody can be modeled as having been administered into the plasma compartment. A portion of the antibody is transferred from the plasma compartment to vascular plasma in each non-lung organ, as indicated by an arrow Q_{org}. From the vascular plasma in each organ to which the antibody has been transferred, the antibody flows into the extravascular space at a flow rate of Q_{org}−L_{org}, while a portion of the antibody is transferred across a vascular wall to interstitial space in the organ by convection and diffusion with a translocation clearance Inflow_{org}. Interstitial fluid flows out from the organ interstitial space at a flow rate of L_{org} and is transferred to the plasma compartment. Depending on the organ, L_{org} and/or Inflow_{org} may be zero.

Each solid or dashed line dividing each organ compartment into two parts represents vascular walls that separate blood vessels from interstitial space in each organ. Highly impermeable vascular walls are indicated by a solid line, and vascular walls that are present but permit relatively easy transfer of the antibody into interstitial space are indicated by a dashed line. An organ without a partition line is regarded as an organ in which the antibody in blood vessels can act substantially directly on tissue cells. In each organ compartment, V_{Vorg} represents a vascular plasma volume, and Vi_{org} represents an interstitial fluid volume.

In FIG. 3, as an example, the heart compartment is illustrated with a plasma volume V_{Vorg}, an interstitial space volume Vi_{org}, a clearance CL_{org} indicating linear elimination of the antibody, Kint_{org} indicating an internalization rate of the antigen and the antigen-antibody complex, Q_{org}−L_{org} indicating the antibody returning via a vein to the lung, and an arrow Inflow_{org} indicating transfer from vascular plasma within each organ to the organ interstitial space across the vascular wall by convection and diffusion. The same applies to the other organs.

From the blood vessels within each organ, linear elimination due to clearance occurs, as indicated by CL_{org}. This clearance corresponds, for example, to an amount eliminated due to non-specific adsorption of the antibody onto a cell surface and subsequent pinocytosis. In certain organs, such as the kidney and the brain, with highly impermeable vascular walls, permeation of the antibody from vascular plasma within the organ into interstitial space is highly restricted; because the amount of permeation into the interstitial space is negligible (or substantially zero), the permeation of the antibody into interstitial space may be neglected. Accordingly, the amount of the antibody in vascular plasma in each organ changes due to inflow from the plasma compartment, outflow to the lung, and clearance; however, translocation clearance to the interstitial space is not taken into account in calculation. For example, when expressing a change in the amount of the antibody in vascular plasma within each organ, the change may be represented by excluding, from the equation, a term representing translocation clearance; alternatively, the calculation may be performed by setting the amount of the translocation clearance to zero. In terms of antibody inflow and outflow, the lung compartment differs from the other organ compartments in that the antibody flows into the lung compartment from each organ and flows out to the plasma compartment.

The change over time in the amount of the antibody in the blood vessels within each organ is represented as a value obtained by subtracting, from an amount of inflow from the plasma compartment, a clearance-mediated elimination amount (organ uptake) in each organ and an amount of outflow to the other organs, such as the lung, due to blood flow. The various amounts of inflow and outflow of the antibody may be calculated by multiplying a blood flow rate by an antibody concentration (obtained by dividing the antibody amount by a volume). When organs such as the kidney and the brain, in which vascular walls and the like function as barriers, are collectively referred to as “Organ1”, the change over time in the amount of the antibody in the blood vessels within each organ can be expressed as the following Formula (1).

$$[ Formula 1 ]$$

$$\frac{{dAp}_{organ 1}}{dt} = \frac{{Q}_{o r g 1} \cdot {Ap}_{p l a s m a}}{{V}_{p l a s m a}} - \frac{A {p}_{o r g a n 1}}{{Vv}_{o r g 1}} \cdot {CL}_{o r g 1} - \frac{{Q}_{o r g 1} \cdot {Ap}_{o r g a n 1}}{{Vv}_{o r g 1}} \qquad ( 1 )$$

In the above Formula (1),
Apₚₗₐₛₘₐ represents the amount of the antibody in the plasma compartment;
Vₚₗₐₛₘₐ represents the volume of the plasma compartment; Ap_{organ1} represents the amount of the antibody in the blood vessels within the organ;
dAp_{organ1}/dt represents the change over time in the amount of the antibody in the blood vessels within the organ per unit time;
Q_{org1} represents the flow rate of the antibody from the plasma compartment;
V_{Vorg1} represents the volume of the vascular plasma within the organ; and CL_{org1} represents clearance-mediated elimination corresponding to cellular uptake (e.g., by vascular endothelial cells) in the organ and subsequent degradation of the antibody.

Alternatively, a term “−Inflow_{org1}×(Ap_{organ1}/V_{Vorg1})” subtracting translocation clearance of an antibody from vascular plasma in the organ to interstitial space of the organ of interest may be added to the right-hand side of Formula (1), and Inflow_{org1} may be assigned a small value, thereby incorporating a negligible translocation clearance into the model.

For each organ, an amount of an antigen (a membrane protein) expressed on cell surfaces is defined. For example, the amount of change in the antigen may be calculated by subtracting, from the amount of antigen of the antigen protein that binds to the antibody, an amount of elimination of antigen protein bound to the antibody and an amount of binding between the antigen protein and the antibody, and further subtracting a dissociation amount of the antibody dissociated from an antigen-antibody complex. The above amount of binding can be calculated from an association rate between the antigen and the antibody. In addition, the above dissociation amount can be calculated from a dissociation rate of the antigen-antibody complex. As one embodiment, in particular, when organs in which the antigen is expressed are collectively referred to as “Organ2”, the following Formula (2) is assigned:

$$[ Formula 2 ]$$

$$\frac{dOrgan2\_Antigen}{dt} = {Organ2\_Ksyn}_{A n t i g e n} - {KinT}_{A n t i g e n} \cdot Organ2\_Antigen - ( \frac{{K}_{o n} \cdot Organ 2. Ag}{V {i}_{org 2}} \cdot \frac{A {i}_{o r g 2}}{V {i}_{o r g 2}} - \frac{{K}_{off} \cdot Organ 2. Comp}{V {i}_{org 2}} ) \cdot {Vi}_{org 2} \qquad ( 2 )$$

In the above Formula (2),
Organ2_Antigen represents the amount of antigen (a membrane protein) expressed on cell surfaces;
dOrgan2_Antigen/dt represents the change over time in the amount of antigen present on cell surfaces;
Organ2_Ksyn_{Antigen} represents the rate of synthesis of the antigen; and
Kint_{Antigen}·Organ2_Antigen represents the rate of elimination of the antigen.

Note that the third term on the right-hand side will be described later with reference to Formula (5).

In organs in which vascular walls are present but transfer of the antibody into interstitial space is relatively easy (two compartments are indicated with dashed lines; e.g., heart), it is necessary to take into account transfer of the antibody from vascular plasma in the organ to interstitial space of the organ. Accordingly, the amount of the antibody in vascular plasma in each organ changes due to inflow from the plasma compartment, clearance in the organ, translocation clearance to interstitial space, and outflow to other organs, such as the lung, due to blood flow. For example, the amount of the antibody in vascular plasma within the organ can be calculated by subtracting, from an amount of inflow of the antibody from the plasma compartment, an amount of outflow to the lung compartment, an amount of transfer to interstitial space within the organ, and clearance. In addition, based on anatomical knowledge, an amount of inflow of the antibody from other organs may be included in certain organs, or alternatively, an amount of outflow of the antibody to other organs may be subtracted in the corresponding equation.

In addition, the amount of outflow may be calculated by subtracting a flow rate of lymph flow returning, via lymphatic vessels, to plasma from an inflow rate from the plasma compartment, and multiplying the resulting net flow rate by the antibody concentration in plasma of the organ compartment. Note that in the case of the lung compartment, the calculation is performed using an amount of inflow from each organ compartment to the lung compartment and an amount of outflow to the plasma compartment. In one embodiment, in the case of an organ in which vascular walls are present but transfer of the antibody into interstitial space is possible, the change over time in the amount of antibody in the blood vessels within each organ is represented by the following Formula (3).

$$[ Formula 3 ]$$

$$\frac{d A {p}_{o r g a n 2}}{dt} = \frac{{Q}_{o r g 2}}{{V}_{P l a s m a}} \cdot {Ap}_{p l a s m a} - \frac{A {p}_{o r g a n 2}}{{Vv}_{o r g 2}} \cdot {CL}_{o r g 2} - \frac{{Inflow}_{o r g 2} \cdot {Ap}_{o r g a n 2}}{{Vv}_{o r g 2}} - \frac{( {Q}_{o r g 2} - {L}_{o r g 2} ) \cdot {Ap}_{o r g a n 2}}{{Vv}_{o r g 1}} \qquad ( 3 )$$

In the above Formula (3),
Ap_{organ2} represents the amount of the antibody in the blood vessels within the organ;
dAp_{organ2}/dt represents the change over time in the amount of the antibody in the blood vessels within the organ per unit time;
Q_{org2} represents the flow rate of plasma from the plasma compartment into vascular plasma within the organ;
L_{org2} represents the lymph flow rate in the organ;
V_{Vorg2} represents the volume of vascular plasma within the organ;
CL_{org2} represents clearance-mediated elimination corresponding to cellular uptake (e.g., by vascular endothelial cells) in the organ and subsequent degradation of the antibody; and
Inflow_{org2} represents translocation clearance into interstitial space of the organ.

The first term in Formula (3) corresponds to transfer of the antibody from the plasma compartment into the blood vessels within the organ. The second term in Formula (3) corresponds to clearance-mediated elimination from vascular plasma within the organ. In addition, the third term in Formula (3) corresponds to transfer from intravascular space within the organ to interstitial space of the organ of interest. The numerator of the fourth term in Formula (3), (Q_{org2}−L_{org2}) represents a net flow rate obtained by subtracting the lymph flow rate from the flow rate of blood to the cardiac vasculature, and corresponds to transfer of the antibody within the blood vessels the organ to the lung.

Here, the antibody transferred to the interstitial space of each organ binds to an antigen expressed on cell membranes of each organ and forms an antigen-antibody complex. After formation of the antigen-antibody complex, a portion of the antibody dissociates from the antigen. Once internalized into cells, the antigen-antibody complex is eliminated due to protease action, etc. The rate of elimination of the amount of the antigen-antibody complex in each organ can be expressed by the following Formula (4).

An object of the present disclosure is to predict the concentration-time profile of an antibody in blood based on a prediction model that reflects in vivo kinetics while ensuring applicability, and to provide a dose-determination tool based on such prediction. However, the model described in NPL 1 does not sufficiently represent the actual in vivo kinetics of an antibody.

[Formula 4]

Kᵢₙₜ·Organ2·Comp (4)

In Formula (4), Kᵢₙₜ represents the internalization rate constant of the antigen-antibody complex, and Organ2.Comp represents the amount of the antigen-antibody complex in the organ. In Formula (4), Kᵢₙₜ may be equal to Kint_{antigen}, which is the internalization rate constant of the antigen internalized into cells.

Based on the above, for example, the amount of change in the antigen-antibody complex is calculated by subtracting, from the amount of the reference antibody bound to the antigen, the amount of dissociation from the antigen and an amount obtained by multiplying the amount of the antigen-antibody complex by the internalization rate. In one embodiment, the change over time (time derivative) in the amount of the antigen-antibody complex in each organ is expressed by the following Formula (5):

$$[ Formula 5 ]$$

$$\frac{dOrgan 2. Comp}{dt} = ( \frac{{K}_{o n} \cdot Organ 2. Ag}{{Vi}_{org 2}} \cdot \frac{{Ai}_{org 2}}{{Vi}_{org 2}} - \frac{{K}_{off} \cdot Organ 2. Comp}{V {i}_{org 2}} ) \cdot {Vi}_{org 2} - Kint \cdot Organ 2. Comp \qquad ( 5 )$$

In Formula (5), dOrgan2.Comp/dt represents the change in the amount of the antigen-antibody complex. Kₒₙ represents the association rate constant; Organ2.Ag represents the amount of antigen expressed on the cell membranes and unbound to the antibody; Vi_{org2} represents the volume of interstitial fluid; Ai_{org2} represents the amount of antibody that is in the interstitial space of the organ and is unbound to the antigen. K_{off} represents the dissociation rate constant; and Organ2.Comp represents the amount of the antigen-antibody complex in the organ. In addition to Formula(5), by combining a balance equation for synthesis and degradation of a target protein on cell surfaces of the organ, the rate of internalization of the complex-forming antibody into cells as well as production and elimination of the target antigen are determined.

In addition, the change over time in the amount of the antibody in interstitial fluid of the organ is determined as a result of subtracting, from the amount of transfer of the antibody due to inflow from vascular plasma within the organ, the amount of binding due to binding to the antigen, then adding the amount of dissociation from the antigen-antibody complex, and further subtracting the transfer amount of the antibody transferred to the plasma compartment via lymphatic return flow. In one embodiment, the change over time in the amount of the antibody in interstitial space within the organ is expressed by the following Formula (6).

$$[ Formula 6 ]$$

$$\frac{d A {i}_{o r g 2}}{d r} = \frac{{Inflow}_{o r g a n 2} \cdot {Ap}_{o r g 2}}{{Vv}_{o r g 2}} - ( \frac{{K}_{o n} \cdot Organ 2. Ag}{{Vi}_{o r g 2}} \cdot \frac{A {i}_{o r g 2}}{{Vi}_{o r g 2}} - \frac{{K}_{off} \cdot Organ 2. Comp}{{Vi}_{o r g 2}} ) \cdot {Vi}_{o r g 2} - \frac{{L}_{org 2} . A {i}_{org 2}}{{Vi}_{o r g 2}} \qquad ( 6 )$$

In the above Formula (6),
Ai_{org2} represents the amount of antibody that is present in interstitial space of the organ and is unbound to the antigen;
dAi_{org2}/dt represents the change over time in the amount of antibody that is present in interstitial space of the organ and is unbound to the antigen;
L_{org2} represents the lymph flow rate;
Kₒₙ represents the association rate constant of the antibody;
K_{off} represents the dissociation rate constant of the antibody;
Organ2.Ag represents the amount of antigen that is expressed on cell membranes and is unbound to the antibody; and
Organ2.Comp represents the amount of the antigen-antibody complex.

In addition, as indicated by the arrow L_{org} in FIG. 3, a pathway is provided in which a portion of the antibody returns from interstitial space of each organ to the plasma compartment, thereby taking into account outflow from interstitial space of the organ via lymphatic flow. Note that in the case of an organ in which the antigen amount is zero, the amount of antigen expressed on the cell membranes may be set to zero, or a formula excluding terms relating to the antigen amount and binding to and dissociation from the antigen may be used.

In organs such as the liver and spleen, where the antibody in plasma can be considered to act substantially directly on tissue cells, calculations may be performed without distinguishing between vascular plasma antibody and interstitial antibody within the organ. The change over time in the amount of the antibody in the organ is expressed as a result of subtracting, from the amount of transfer of the antibody from the plasma compartment to the organ, the amount of transfer to the lung compartment, the amount of elimination due to non-specific clearance, and the amount of elimination due to binding to the antigen, and adding the amount of dissociation of the antigen-antibody complex. Depending on anatomical structure, inflow of the antibody from the other organ compartments may be added, or outflow of the antibody to the other organ compartments may be subtracted. When organs in which the antibody in vascular plasma within the organ can be regarded as acting substantially directly on tissue cells are collectively referred to as “Organ3”, the change over time in the antibody amount in the organ can be expressed as the following Formula (7).

$$[ Formula 7 ]$$

$$\frac{d A {p}_{o r g a n 3}}{d r} = \frac{{Q}_{o r g 3} \cdot {Ap}_{p ι a s m a}}{{V}_{p l a s m a}} - \frac{A {p}_{o r g a n 3}}{{V}_{{V}_{O r g 3}}} \cdot {CL}_{o r g 3} - ( \frac{{K}_{o n} \cdot Organ 3. Ag}{{V}_{{V}_{O r g 3}}} \cdot \frac{A {p}_{o r g 3}}{{V}_{{V}_{O r g 3}}} - \frac{{K}_{off} \cdot Organ 3. Comp}{{V}_{{V}_{O r g 3}}} ) \cdot {V}_{{V}_{O r g 3}} - \frac{{Q}_{o r g 3} \cdot {Ap}_{o r g a n 3}}{{Vv}_{o r g 3}} \qquad ( 7 )$$

In the above Formula (7),
Ap_{organ3} represents the amount of the antibody that is present in the organ and is unbound to the antigen;
Organ3.Comp represents the amount of the antigen-antibody complex within the organ;
Ap_{organ3}/dt represents the change over time in the amount of antibody that is present in the organ and is unbound to the antigen;
Q_{org3} represents the flow rate of blood transferred from the plasma compartment into the organ; V_{Vorg3} represents the sum of the volumes of plasma and interstitial fluid in the organ;
CL_{org3} represents clearance-mediated elimination in the organ;
Kₒₙ represents the association rate constant between the antigen and the antibody;
K_{off} represents the dissociation rate constant between the antigen and the antibody; and
Organ3.Ag represents the amount of antigen that is expressed on cell surfaces and is unbound to the antibody.

Further, when an organ receives blood inflow from other organs, as in the case of the liver receiving blood from the spleen and gastrointestinal tract, the amount of inflow to the target organ may be calculated by summing the amounts of the antibody flowing out from the other organs into the target organ. Even in Organ3, where the antibody in plasma is considered to act substantially directly on tissue cells, association and dissociation between the antibody and the antigen are taken into account via the third and fourth terms of Formula (7), and the change in the amount of the antigen-antibody complex is governed by Formula (5), which incorporates antibody elimination due to internalization.

An antibody capable of binding to an antigen expressed on cell membranes may be administered. At that time, the rate of elimination of the antibody may accelerate at a certain concentration of the antibody in plasma. In this case, it has been known that the dose of the antibody is not proportional to its exposure. The fact that the antibody dose is not proportional to the exposure makes prediction of the exposure difficult, thereby presenting a challenge in drug development. It is known that one of the causes of a marked decrease in antibody concentration after administration is internalization of the antigen-antibody complex into cells. The prediction model according to this embodiment enables prediction of a time profile of the concentration of the antibody in a non-linear elimination range by taking into account internalization of the antigen-antibody complex into cells.

In addition, in this embodiment, when setting the PBPK model parameters involving antibody inflow to each organ and plasma, antibody outflow from each organ and plasma, and antibody metabolism in each organ and plasma as well as including the rate of internalization of the antigen-antibody complex into cells, it is necessary to more accurately identify the amount and expression level of a protein of interest in each organ. In this embodiment, the amounts of proteins and their metabolic rates in each organ are identified by proteomics analysis and incorporated into parameters of the prediction model, thereby enabling further improvement in prediction accuracy of the multi-compartment prediction model.

The physiological pharmacokinetics of the antibody have been explained from three viewpoints of multiple organs-Organ1, Organ2, and Organ3-according to differences in vascular wall structure among organs, including whether the antibody in vascular plasma of the organ permeates into the interstitial space. Here, the physiological pharmacokinetics of an antibody unbound to its antigen in a plurality of specific organs adopted in the prediction model shown in FIG. 3 are expressed by the following differential equations.

$$[ Formula 8 ‐ 1 ]$$

$$\frac{d ( A {p}_{p l a s m a} )}{dt} = \frac{( {Q}_{l u n} - {L}_{l u n} ) \cdot {Ap}_{l u n g}}{V {v}_{l u n}} + \frac{{L}_{l u n} \cdot {Ai}_{l u n g}}{V {i}_{l u n}} - \frac{{Q}_{b o n} \cdot {Ap}_{p l a s m a}}{V {v}_{p l a}} - \frac{{Q}_{a l i v} \cdot {Ap}_{p l a s m a}}{V {v}_{p l a}} - \frac{{Q}_{s k i} \cdot {Ap}_{p l a s m a}}{V {v}_{p l a}} + \frac{{L}_{s k i} \cdot {Ai}_{s k i n}}{V {i}_{s k i}} - \frac{{Q}_{m u s} \cdot {Ap}_{p l a s m a}}{V {v}_{p l a}} + \frac{{L}_{m u s} \cdot {Ai}_{m u s c l e}}{V {i}_{m u s}} - \frac{{Q}_{s p l} \cdot {Ap}_{p l a s m a}}{V {v}_{p l a}} - \frac{{Q}_{g u t} \cdot {Ap}_{p l a s m a}}{V {v}_{p l a}} + \frac{{L}_{g u t} \cdot {Ai}_{gitract}}{V {i}_{g u t}} - \frac{{Q}_{kid} \cdot {Ap}_{plasma}}{V {v}_{pla}} + \frac{{L}_{kid} \cdot {Ai}_{kidney}}{V {i}_{kid}} - \frac{{Q}_{h e a} \cdot {Ap}_{plasma}}{V {v}_{pla}} + \frac{{L}_{h e a} \cdot {Ai}_{heart}}{V {i}_{h e a}} - \frac{{Q}_{adi} \cdot {Ap}_{plasma}}{V {v}_{pla}} + \frac{{L}_{adi} \cdot {Ai}_{adipose}}{V {i}_{adi}} - \frac{{Q}_{bra} \cdot {Ap}_{plasma}}{V {v}_{pla}} + \frac{{L}_{bra} \cdot {Ai}_{brain}}{V {i}_{bra}} - \frac{C {L}_{o thers} \cdot {Ap}_{plasma}}{V {v}_{pla}} \qquad ( a )$$

$$\frac{d ( A {p}_{lung} )}{d T} = - \frac{{Inflow}_{lun} \cdot {Ap}_{lung}}{V {v}_{lun}} - \frac{( {Q}_{lun} - {L}_{lun} ) \cdot {Ap}_{lung}}{V {v}_{lun}} + \frac{{Q}_{b o n} \cdot {Ap}_{b o n e}}{V {v}_{b o n}} + \frac{( {Q}_{aliv} + {Q}_{s p i} + {Q}_{g u t} - {L}_{g u t} ) \cdot {Ap}_{liver}}{V {v}_{liv}} + \frac{( {Q}_{s k i} - {L}_{s k i} ) \cdot {Ap}_{s k i n}}{{Vv}_{s k i}} + \frac{( {Q}_{m u s} - {L}_{m u s} ) \cdot {Ap}_{m u s c ι e}}{V {v}_{m u s}} + \frac{{Q}_{kid} \cdot {Ap}_{kidney}}{V {v}_{kid}} + \frac{( {Q}_{h e a} - {L}_{h e a} ) \cdot {Ap}_{heart}}{V {v}_{h e a}} + \frac{( {Q}_{adi} - {L}_{adi} ) \cdot {Ap}_{adipose}}{V {v}_{adi}} + \frac{{Q}_{bra} \cdot {Ap}_{brain}}{V {v}_{bra}} - \frac{A {p}_{lung}}{V {v}_{lun}} \cdot {CL}_{lun} \qquad ( b )$$

$$[ Formula 8 ‐ 2 ]$$

$$\frac{d ( A {i}_{l u n g} )}{d t} = \frac{{Inflow}_{l u n} \cdot {Ap}_{l u n g}}{V {v}_{l u n}} - \frac{{L}_{l u n} \cdot {Ai}_{l u n g}}{V {i}_{l u n}} \qquad ( c )$$

$$\frac{d ( A {p}_{b o n e} )}{d t} = \frac{{Q}_{b o n} \cdot {Ap}_{p l a s m a}}{V {v}_{p l a}} - \frac{{Q}_{b o n} \cdot {Ap}_{b o n e}}{V {v}_{b o n}} - \frac{A {p}_{b o n e}}{V {v}_{b o n}} \cdot {CL}_{b o n} \qquad ( d )$$

$$\frac{d ( A {p}_{liver} )}{dt} = \frac{{Q}_{aliv} \cdot {Ap}_{plasma}}{V {v}_{pla}} - \frac{( {Q}_{aliv} + {Q}_{s p l} + {Q}_{g u t} - {L}_{g u t} ) \cdot {Ap}_{liver}}{V {v}_{liv}} + \frac{{Q}_{spi} \cdot {Ap}_{spleen}}{V {v}_{spl}} + \frac{( {Q}_{g u t} - {L}_{g u t} ) \cdot {Ap}_{gitract}}{V {v}_{g u t}} - \frac{A {p}_{liver}}{V {v}_{liv}} \cdot {CL}_{liv} \qquad ( e )$$

$$\frac{d ( A {p}_{s k i n} )}{d t} = \frac{{Q}_{s k i} \cdot {Ap}_{plasma}}{V {v}_{pla}} - \frac{{Inflow}_{s k i} \cdot {Ap}_{s k i n}}{V {v}_{s k i}} - \frac{( {Q}_{s k i} - {L}_{s k i} ) \cdot {Ap}_{s k i n}}{V {v}_{s k i}} - \frac{A {p}_{s k i n}}{V {v}_{s k i}} \cdot {CL}_{s k i} \qquad ( f )$$

$$\frac{d ( A {i}_{s k i n} )}{d t} = \frac{{Inflow}_{s k i} \cdot {Ap}_{s k i n}}{V {v}_{s k i}} - \frac{{L}_{s k i} \cdot {Ai}_{s k i n}}{V {i}_{s k i}} \qquad ( g )$$

$$[ Formula 8 ‐ 3 ]$$

$$\frac{d ( A {p}_{m uscle} )}{d t} = \frac{{Q}_{m u s} \cdot {Ap}_{plasma}}{{Vv}_{pla}} - \frac{{Inflow}_{m u s} \cdot {A}_{P m uscle}}{V {v}_{m u s}} - \frac{( {Q}_{m u s} - {L}_{m u s} ) \cdot {Ap}_{m u s c ι e}}{V {v}_{m u s}} - \frac{A {p}_{m u s c ι e}}{V {v}_{m u s}} \cdot {CL}_{m u s} \qquad ( h )$$

$$\frac{d ( A {i}_{m uscle} )}{d t} = \frac{{Inflow}_{m u s} \cdot {A}_{P m u s c l e}}{V {v}_{m u s}} - \frac{{L}_{m u s} \cdot {Ai}_{m u s c ι e}}{V {i}_{m u s}} \qquad ( i )$$

$$\frac{d ( A {p}_{s p l e e n} )}{d t} = \frac{{Q}_{spl} \cdot {Ap}_{plasma}}{V {v}_{pla}} - \frac{{Q}_{spl} \cdot {Ap}_{spleen}}{V {v}_{spl}} - \frac{A {p}_{spleen}}{V {v}_{spl}} \cdot {CL}_{s p l} \qquad ( j )$$

$$\frac{d ( A {p}_{g i t r a c t} )}{d t} = \frac{{Q}_{g u t} \cdot {Ap}_{plasma}}{{Vv}_{pla}} - \frac{{Inflow}_{g u t} \cdot {Ap}_{gitract}}{{Vv}_{g u t}} - \frac{( {Q}_{g u t} - {L}_{g u t} ) \cdot {Ap}_{g i t r a c t}}{{Vv}_{g u t}} - \frac{A {p}_{g i t r a c t}}{{Vv}_{g u t}} \cdot {CL}_{g u t} \qquad ( k )$$

$$\frac{d ( A {i}_{g i t r a c t} )}{d t} = \frac{{Inflow}_{gut} \cdot {Ap}_{gitract}}{{Vv}_{m u s}} - \frac{{L}_{g u t} \cdot {Ai}_{g i t r a c t}}{{Vi}_{g u t}} \qquad ( l )$$

$$[ Formula 8 ‐ 4 ]$$

$$\frac{d ( A {p}_{kidney} )}{d t} = \frac{{Q}_{kid} \cdot {Ap}_{plasma}}{V {v}_{pla}} - \frac{{Q}_{kid} \cdot {Ap}_{kidney}}{V {v}_{kid}} - \frac{A {p}_{kidney}}{V {v}_{kid}} \cdot {CL}_{kid} \qquad ( m )$$

$$\frac{d ( A {p}_{h e a r t} )}{d t} = \frac{{Q}_{h e a} \cdot {Ap}_{plasma}}{{Vv}_{pla}} - \frac{{Inflow}_{h e a} \cdot {Ap}_{h e a r t}}{V {v}_{h e a}} - \frac{( {Q}_{h e a} - {L}_{h e a} ) \cdot {Ap}_{h e a r t}}{V {v}_{h e a}} - \frac{A {p}_{h e a r t}}{V {v}_{h e a}} \cdot {CL}_{h e a} \qquad ( n )$$

$$\frac{d ( A {i}_{h e a r t} )}{d t} = \frac{{Inflow}_{h e a} \cdot {Ap}_{h e a r t}}{V {v}_{h e a}} - \frac{{L}_{h e a} \cdot {Ai}_{h e a r t}}{V {i}_{h e a}} \qquad ( o )$$

$$\frac{d ( A {p}_{adipose} )}{d t} = \frac{{Q}_{adi} \cdot {Ap}_{plasma}}{{Vv}_{pla}} - \frac{{Inflow}_{adi} \cdot {Ap}_{adipose}}{{Vv}_{adi}} - \frac{( {Q}_{adi} - {L}_{adi} ) \cdot {Ap}_{adipose}}{{Vv}_{adi}} - \frac{A {p}_{adipose}}{{Vv}_{adi}} \cdot {CL}_{adi} \qquad ( p )$$

$$\frac{d ( A {i}_{adipose} )}{d t} = \frac{{Inflow}_{adi} \cdot {Ap}_{adipose}}{{Vv}_{adi}} - \frac{{L}_{adi} \cdot {Ai}_{adipose}}{{Vi}_{adi}} \qquad ( q )$$

$$\frac{d ( A {p}_{b r a i n} )}{d t} = \frac{{Q}_{b r a} \cdot {Ap}_{plasma}}{{Vv}_{pla}} - \frac{{Q}_{b r a} \cdot {Ap}_{b r a i n}}{{Vv}_{b r a}} - \frac{A {p}_{b r a i n}}{{Vv}_{b r a}} \cdot {CL}_{b r a} \qquad ( r )$$

In Equations (a) to (r), Apₚₗₐₛₘₐ represents the amount of the antibody in the plasma compartment, and Ap_{lung}, Ap_{bone}, Apₗᵢᵥₑᵣ, Apₛₖᵢₙ, Apₕₑₐᵣₜ, Ap_{muscle}, Apₛₚₗₑₑₙ, Ap_{gitract}, Ap_{kidney}, Ap_{adipose}, and Ap_{brain} each represent the amount of the antibody in the blood vessels within each corresponding organ. Qₗᵤₙ, Q_{bon}, Qₐₗᵢᵥ, Qₛₖᵢ, Qₕₑₐ, Qₘᵤₛ, Qₛₚₗ, Q_{gut}, Q_{kid}, Q_{adi}, and Q_{bra} each represent the flow rate of blood to each corresponding organ.

In Equations (a) to (r), V_{Vpla} represents the volume of the plasma compartment; and V_{Vlun}, V_{Vbon}, V_{Vliv}, V_{Vski}, V_{Vhea}, V_{Vmus}, V_{Vspi}, V_{Vgut}, V_{Vkid}, V_{Vadi}, and V_{Vbra} each represents the volume of vascular plasma in each corresponding organ. Lₗᵤₙ, Lₛₖᵢ, Lₘᵤₛ, L_{gut}, Lₕₑₐ, and L_{adi} each represent the flow rate of lymphatic flow from each corresponding organ. Ai_{lung}, Aiₛₖᵢₙ, Aiₕₑₐᵣₜ, Ai_{muscle}, Ai_{gitract}, and Ai_{adipose} each represent the amount of the antibody in interstitial fluid of each corresponding organ.

In Equations (a) to (r), Viₗᵤₙ, Viₛₖᵢ, Viₕₑₐ, Viₘᵤₛ, Vi_{gut}, and Vi_{adi} each represent the volume of interstitial fluid in each corresponding organ. CLₗᵤₙ, CL_{bon}, CLₗᵢᵥ, CLₛₖᵢ, CLₕₑₐ, CLₘᵤₛ, CLₛₚᵢ, CL_{gut}, CL_{kid}, CL_{adi}, CL_{bra}, and CLₒₜₕₑᵣₛ each represent antibody clearance in each corresponding organ. In Equations (a) to (r), Inflowₗᵤₙ, Inflowₛₖᵢ, Inflowₘᵤₛ, Inflow_{gut}, Inflowₕₑₐ, and Inflow_{adi} each represent translocation clearance of the antibody into the interstitial space of each corresponding organ.

Equation (a) expresses the change over time in the antibody amount in the plasma compartment. Equation (b) expresses the change over time in the antibody amount in the blood vessels in the lung. Equation (c) expresses the change over time in the antibody amount in the interstitial space of the lung. Equation (d) expresses the change over time in the antibody amount in the blood vessels in the bone. Equation (e) expresses the change over time in the antibody amount in the blood vessels of the liver. Equation (f) expresses the change over time in the antibody amount in the blood vessels of the skin. Equation (g) expresses the change over time in the antibody amount in the interstitial space of the skin. Equation (h) expresses the change over time in the antibody amount in the blood vessels of the muscle.

Equation (i) expresses the change over time in the antibody amount in the interstitial space of the muscle. Equation (j) expresses the change over time in the antibody amount in the blood vessels of the spleen. Equation (k) expresses the change over time in the antibody amount in the blood vessels of the gastrointestinal tract. Equation (1) expresses the change over time in the antibody amount in the interstitial space of the gastrointestinal tract. Equation (m) expresses the change over time in the antibody amount in the blood vessels of the kidney. Equation (n) expresses the change over time in the antibody amount in the blood vessels of the heart.

Equation (o) expresses the change over time in the antibody amount in the interstitial space of the heart. Equation (p) expresses the change over time in the antibody amount in the blood vessels of the adipose tissue. Equation (q) expresses the change over time in the antibody amount in the interstitial space of the adipose tissue. Equation (r) expresses the change over time in the antibody amount in the blood vessels of the brain.

When the lung is taken as an example of the organ compartments constituting the prediction model shown in FIG. 3, the rate of non-specific elimination of the antibody in the lung is expressed by the last term of Equation (b) above.

$$\begin{matrix}[ Formula 9 ] & \\ \frac{A {p}_{l u n g}}{{Vv}_{l u n}} . C {L}_{l u n} & ( 8 )\end{matrix}$$

That is, the rate of non-specific elimination of the antibody in the lung is obtained by multiplying the antibody concentration in the blood vessels of the lung by the clearance-mediated elimination in the lung.

### [Functional Blocks of Prediction Apparatus]

The prediction apparatus 1 includes a memory (memory unit) 10, an input unit 11 (also referred to herein as input circuitry), a prediction unit 12 (also referred to herein as prediction circuitry), and a presentation unit 13 (also referred to herein as presentation circuitry). As used herein, the term “circuitry” refers broadly to hardware that performs the recited functions, including general purpose processors executing programs stored in memory, dedicated circuits (such as ASICs and FPGAs), and combinations thereof. As used herein, the memory (memory unit) 10, input unit 11, prediction unit 12, and presentation unit 13 may be implemented as separate circuits or as combined circuitry; the term “prediction circuitry” encompasses any circuitry that performs one or more of the recited functions. More generally, references to a “unit” in this disclosure encompass any hardware element or combination of hardware elements that performs the described function.

Next, the configuration of a prediction apparatus 1 included in the prediction system will be described with reference to the block diagram of FIG. 2. As shown in FIG. 2, the prediction apparatus 1 includes a memory (memory unit) 10, an input unit 11, a prediction unit 12, and a presentation unit 13. The prediction apparatus 1 predicts a time profile of the concentration of a prediction target antibody by using a prediction model constructed in advance.

The memory 10 stores a prediction program 10A and a prediction model 10B. The prediction program 10A is a program including a plurality of instructions for applying the prediction model based on input information on a prediction target antibody, performing computational processing, and outputting a prediction result. The prediction model 10B is a prediction model constructed in advance by a model construction apparatus 2 described later. The prediction apparatus 1 is capable of receiving the prediction model from the model construction apparatus 2 via a communication interface 104. The memory 10 stores information indicating which antigen protein a preset prediction target antibody binds to, and binding affinity indicating affinity to the antigen protein.

The input unit 11 is configured to receive input information including a dose of the prediction target antibody. More specifically, the input unit 11 may acquire input information other than the initial dose of the prediction target antibody, the information being stored in advance in the memory 10.

The prediction unit 12 is configured to calculate a predicted time profile of the concentration of the prediction target antibody in plasma by using a prediction model. The prediction unit 12 retrieves, from the memory 10, information indicating which antigen protein the input prediction target antibody binds to and a binding affinity indicating affinity to the antigen protein, and automatically sets parameters of the prediction model corresponding to the prediction target antibody. The prediction unit 12 calculates a predicted time profile of the concentration of the antibody in plasma by solving the differential equations represented by Equations (a) to (r).

In some embodiments, the circuitry of the prediction apparatus 1 is further configured to determine, based on the predicted time profile of the plasma concentration of the prediction target antibody, a dose of the prediction target antibody for administration to the subject. For example, the circuitry may compare the predicted time profile against one or more therapeutic criteria (such as a target plasma concentration range, a minimum effective concentration, or a maximum tolerated concentration) and determine a dose that satisfies the therapeutic criteria. The determined dose may be output to the user via the presentation unit 13 (presentation circuitry) or to an external device.

The presentation unit 13 is configured to present the predicted time profile of the concentration of the prediction target antibody in plasma as calculated by the prediction unit 12. The presentation unit 13 can display the predicted profile on a display 107. For example, the presentation unit 13 may display the predicted profile in a graph in which time is plotted on the horizontal axis and the predicted plasma antibody amount is plotted on the vertical axis; however, other display formats may also be employed. The presentation unit 13 may display various parameters in addition to the predicted profile.

### [Functional Blocks of Model Construction Apparatus]

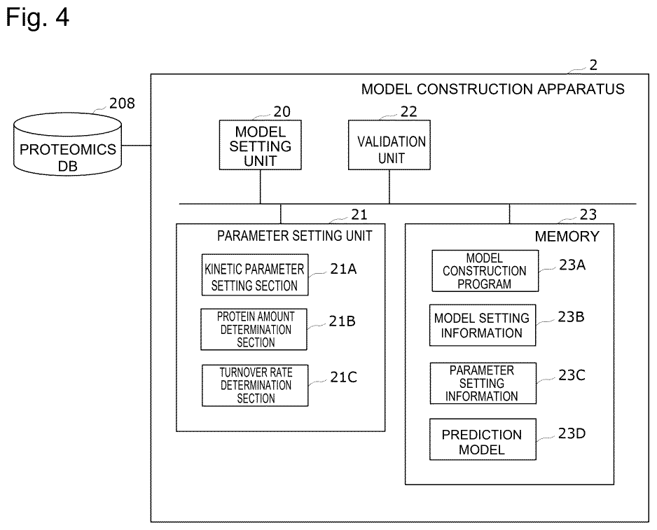

Next, the configuration of the model construction apparatus 2 included in the prediction system will be described with reference to the block diagram in FIG. 4. As shown in FIG. 4, the model construction apparatus 2 includes a model setting unit 20, a parameter setting unit 21, a validation unit 22, and a memory 23. The model construction apparatus 2 is connected to a proteomics database 208 external to the model construction apparatus 2; however, the apparatus need not be connected thereto as long as a means for transferring data is provided. The model construction apparatus 2 is configured to construct a prediction model based on pharmacokinetic data of an antibody (reference antibody). Note that in this embodiment, the model construction apparatus 2 includes the validation unit 22 in order to check the accuracy of the prediction model; however, since the validation unit 22 is not an essential component for estimating the pharmacokinetic concentration of the antibody, the prediction model may be constructed without the validation unit 22.

The model setting unit 20 is configured to set a prediction model. The model setting unit 20 selects, based on a physiologically based pharmacokinetic (PBPK) model, a plasma compartment and a plurality of organ compartments including the lung, heart, brain, kidney, muscle, bone, skin, gastrointestinal tract, liver, spleen, and adipose tissue as shown in FIG. 3, and defines connections among these compartments.

As shown in FIG. 4, the model setting unit 20 is configured to set a prediction model with a pathway in which it is assumed that an administered antibody is administered into the plasma compartment, and in which a portion of the antibody transferred from the plasma compartment to a plurality of organ compartments passes through organ compartments other than the lung and returns to the lung compartment. In addition, the model setting unit 20 sets the type of the antibody. The model setting unit 20 selects an antibody having a nonlinear elimination range in the time profile of the concentration of the antibody, and may, for example, select IgG antibody subclass IgG1, IgG2, IgG3, or IgG4 as the antibody. The pharmacokinetics of IgG subclasses are the same.

The parameter setting unit 21 sets parameters in the prediction model. The parameter setting unit 21 includes a kinetic parameter setting section 21A, a protein amount determination section 21B, and a turnover rate determination section 21C. The kinetic parameter setting section 21A of the parameter setting unit 21 sets, as parameters, inflow of the antibody into the plasma compartment or each of the organ compartments shown in FIG. 3, outflow of the antibody from the plasma compartment or each of the organ compartments, and elimination of the antibody in the plasma compartment or each of the organ compartments.

The kinetic parameter setting section 21A determines, as parameters indicating antibody inflow, an amount of the administered antibody, a rate at which the antibody transfers from plasma into the blood vessels within each organ other than the lung among the plurality of organs, a rate at which the reference antibody transfers from the blood vessels within each organ other than the lung among the plurality of organs to the lung, and a rate at which the antibody transfers from the lung to plasma. The kinetic parameter setting section 21A may set a distribution ratio of the antibody on the basis of the organ size, the blood flow rate, and the like. Alternatively, the kinetic parameter setting section 21A may use an experimentally determined ratio at which the antibody is distributed to each organ.

The kinetic parameter setting section 21A sets, as parameters indicating antibody outflow, clearance of the antibody in plasma and clearance of the antibody in each of the plurality of organs. The kinetic parameter setting section 21A sets, as clearance of the antibody from the blood vessels within each organ, a value corresponding to an amount at which the antibody is eliminated due to non-specific adsorption onto cell surfaces and subsequent processes such as pinocytosis. The kinetic parameter setting section 21A determines clearance of the antibody in plasma and clearance of the antibody in each of the plurality of organs on the basis of physiologically measured results.

The kinetic parameter setting section 21A sets, as parameters indicating antibody elimination, the type and amount of protein capable of binding to the antibody in each of the plurality of organs and plasma. Further, the kinetic parameter setting section 21A sets, as a parameter indicating antibody elimination, the rate at which an antigen-antibody complex, formed by binding of the antibody transferred from plasma to each of the plurality of organs to a protein present in each corresponding organ, is internalized into cells of each corresponding organ. Furthermore, the kinetic parameter setting section 21A sets, as a parameter indicating antibody elimination, an association rate constant (Kₒₙ) representing a rate at which the antibody transferred to each of the plurality of organs binds to a protein present in each corresponding organ. In addition, the parameter setting unit 21 further sets, as a parameter indicating antibody kinetics, a dissociation rate constant (K_{off}) at which the antigen-antibody complex dissociates into the antibody and the protein in plasma or in each of the plurality of organs.

Furthermore, the kinetic parameter setting section 21A sets a rate at which the antibody transfers across blood vessels to interstitial space in each of the plurality of organs, and a rate at which the antibody transfers from the interstitial space to the blood vessels in each corresponding organ. The kinetic parameter setting section 21A also determines, based on the concentration of the antibody in interstitial space of each of the plurality of organs, the concentration of the antigen protein that is expressed on cell membranes and is unbound to the antibody, and an association rate, an amount of binding between the antibody transferred to each organ and the antigen protein present in each corresponding organ. In addition, the kinetic parameter setting section 21A determines, based on the amount of the antigen-antibody complex and the internalization rate, an amount at which the antigen-antibody complex is internalized into cells of each of the plurality of organs and eliminated.

The kinetic parameter setting section 21A determines, based on the amount of the antibody in interstitial space of each of the plurality of organs and a lymph flow rate in each of the plurality of organs, an amount of the antibody transferred from the interstitial space to plasma. The kinetic parameter setting section 21A also determines, based on the amount of the antibody in interstitial space of each of the plurality of organs and a rate at which the antibody transferred to each organ binds to the antigen protein present in each corresponding organ, an amount of binding between the antibody transferred to each organ and the protein present in each corresponding organ. Furthermore, the kinetic parameter setting section 21A determines, based on a rate at which the antigen-antibody complex dissociates into the antibody and the protein in plasma and in each of the plurality of organs, an amount of the antibody dissociating from the protein in each of the plurality of organs.

The kinetic parameter setting section 21A can set the above-described parameters for organs having highly impermeable vascular walls, such as the kidney and the brain, in accordance with Formula (1). In addition, the kinetic parameter setting section 21A can set the respective parameters, in accordance with Formulas (2) to (6), for organs in which vascular walls are present but the antibody is capable of transferring into interstitial space of the organ, namely the lung, heart, muscle, bone, skin, gastrointestinal tract, and adipose tissue. Furthermore, for organs in which the antibody in plasma are considered to act substantially directly on tissue cells, namely the liver and the spleen, the kinetic parameter setting section 21A can set the respective parameters in accordance with Formula (7), and additionally in accordance with Formulas (4) and (5) about the internalization rate. Moreover, the parameter setting unit 21 can ultimately construct the prediction model represented by Equations (a) to (r) in the form of differential equations.

Hereinafter, the configuration of the protein amount determination section 21B will be described in more detail. The protein amount determination section 21B can identify and quantify the levels of proteins expressed in each organ by proteomics analysis. The protein amount determination section 21B is configured to digest proteins derived from each organ with a protease, separate the resulting peptides by liquid chromatography (LC), and analyze the peptides using a mass spectrometer to identify proteins expressed in each organ. The protein amount determination section 21B also performs label-free quantification using data acquired by the mass spectrometer, and calculates an intensity-based absolute quantification (iBAQ) value representing a proportion of a target protein in each organ. The protein amount determination section 21B may acquire, with reference to the proteomics database 208, a proportion of a target protein relative to a total amount of proteins in each organ.

The prediction apparatus 1 includes a memory (memory unit) 10, an input unit 11 (also referred to herein as input circuitry), a prediction unit 12 (also referred to herein as prediction circuitry), and a presentation unit 13 (also referred to herein as presentation circuitry). As used herein, the term “circuitry” refers broadly to hardware that performs the recited functions, including general purpose processors executing programs stored in memory, dedicated circuits (such as ASICs and FPGAs), and combinations thereof. As used herein, the memory (memory unit) 10, input unit 11, prediction unit 12, and presentation unit 13 may be implemented as separate circuits or as combined circuitry; the term “prediction circuitry” encompasses any circuitry that performs one or more of the recited functions. More generally, references to a “unit” in this disclosure encompass any hardware element or combination of hardware elements that performs the described function.

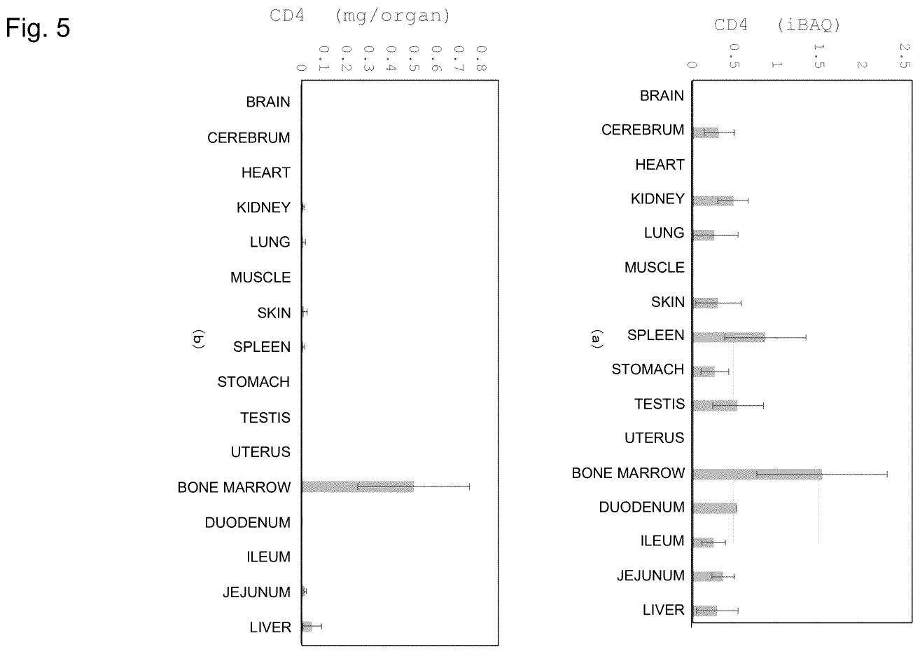

FIG. 5(a) is a diagram showing a proportion [iBAQ] of a target protein relative to a total protein amount in each organ, as identified by LC-MS in the protein amount determination section 21B.

The protein amount determination section 21B can estimate a protein expression level R [nmole] in each organ by using the following Formula (9):

R=[Total protein concentration in the organ]×[Proportion of the target protein relative to the total protein in the organ]×[Volume of the organ] (9).

In Formula (9), the first term is a value obtained by quantification, and the second term is a value obtained by dividing, the value determined from proteomics data by the protein amount determination section 21B as shown in FIG. 5(a) by 1×10⁶. For the third term, a known value of the organ volume obtained from literature or other sources is substituted. In this manner, the protein amount determination section 21B can determine, from the proteomics data, the amount of protein in each organ.

In some embodiments, the circuitry of the prediction apparatus 1 is further configured to determine, based on the predicted time profile of the plasma concentration of the prediction target antibody, a dose of the prediction target antibody for administration to the subject. For example, the circuitry may compare the predicted time profile against one or more therapeutic criteria (such as a target plasma concentration range, a minimum effective concentration, or a maximum tolerated concentration) and determine a dose that satisfies the therapeutic criteria. The determined dose may be output to the user via the presentation unit 13 (presentation circuitry) or to an external device.

The turnover rate determination section 21C can estimate the protein turnover rates by proteomics analysis. The turnover rate determination section 21C may estimate the turnover rate (Kᵢₙₜ) by identifying a protein while using TMT-SILAC (Tandem Mass Tags and Stable Isotope Labeling in Cell Culture) and quantifying de novo synthesis and degradation of the protein. That is, the turnover rate determination section 21C can quantify time-course changes using Tandem Mass Tags (TMT), and can also distinguish and quantify de novo synthesis and degradation of the protein by using stable isotope-labeled amino acids (SILAC). Alternatively, the turnover rate determination section 21C may estimate the protein turnover rate by a SILK (Stable Isotope Labeling Kinetics) method.

The kinetic parameter setting section 21A determines, as a rate at which the antigen-antibody complex is internalized, the protein turnover rate in each organ as determined by the turnover rate determination section 21C. The internalization rate varies depending on the type of the target protein. Meanwhile, the kinetic parameter setting section 21A sets an amount of the target protein in each organ as determined by the protein amount determination section 21B, an association rate (Kₒₙ) at which the antibody binds to the target protein present in each of the plurality of organs, and a dissociation rate (K_{off}) at which the antigen-antibody complex dissociates into the antibody and the target protein in each organ.

In an embodiment with the validation unit 22, the validation unit 22 is configured to validate the prediction model whose parameters have been optimized. The validation unit 22 evaluates prediction accuracy of the constructed prediction model by means of Absolute Average Fold Error (AAFE), comparing it with experimental data. The validation unit 22 may determine that the prediction accuracy is satisfactory when the AAFE is from 1 to 2. In this embodiment, the validation unit 22 validated the prediction model constructed for an antibody targeting the CD4 antigen.

The memory 23 stores a model construction program 23A, model setting information 23B, parameter setting information 23C, and a prediction model 23D. The model construction program 23A is a program including a plurality of instructions in combination for specifying parameters and for validating the prediction model. The model setting information 23B is information about the configuration of the prediction model set by the model setting unit 20. The parameter setting information 23C is information about parameters of the prediction model as set by the parameter setting unit 21. The prediction model 23D is a validated prediction model and includes a system of differential equations represented by Equations (a) to (r) with the set parameters.

### [Hardware Configuration of Prediction Apparatus]

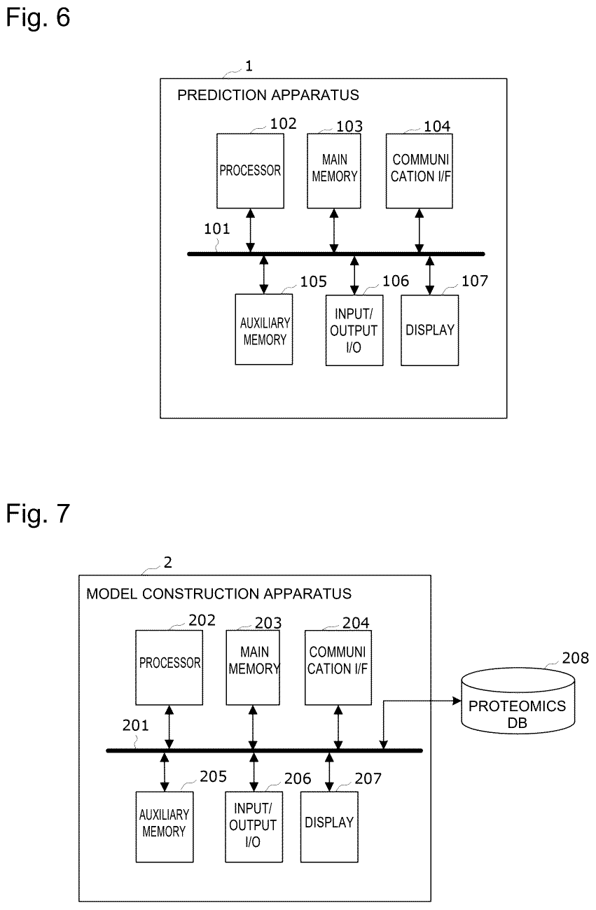

Next, an example of a hardware configuration for implementing the prediction apparatus 1 with the above-described functions will be described with reference to the block diagram of FIG. 6.

As shown in FIG. 6, the prediction apparatus 1 can be implemented by a computer including, for example, a processor 102, a main memory 103, a communication interface (I/F) 104, an auxiliary memory 105, and an input/output (I/O) 106 connected via a bus 101, and by a program that controls these hardware resources. In addition, the prediction apparatus 1 is connected, via the bus 101, to a display 107.

The processor 102 may be implemented by a CPU, a GPU, an FPGA, an ASIC, and/or the like.

The main memory 103 stores, in advance, programs for causing the processor 102 to perform various control operations and calculations. The main memory 103 may be implemented using, for example, a RAM. The processor 102 and the main memory 103 cooperate to implement the respective functions of the prediction apparatus 1, including, for example, the input unit 11 and the prediction unit 12 shown in FIG. 2.

The communication interface (I/F) 104 is an interface circuit configured to establish a network connection between the prediction apparatus 1 and various external electronic devices.

The auxiliary memory 105 includes a readable and writable storage medium and a drive device configured to read and write various types of information, such as programs and data, to and from the storage medium. The auxiliary memory 105 may use, as the storage medium, a hard disk, a solid-state drive (SSD), a ROM, a flash memory, or other semiconductor memories. The auxiliary memory 105 includes a non-transitory storage medium.

The memory 10 described with reference to FIG. 2 is implemented by the auxiliary memory 105. The auxiliary memory 105 may include a program storage area that stores the prediction program 10A to be executed by the prediction apparatus 1. The auxiliary memory 105 may further include, for example, a backup area for backing up the above-described data and programs, or the like.

The input/output (I/O) 106 includes input/output terminals configured to receive signals from external devices and to transmit signals to the external devices. At least part of the configuration of the input unit 11 described with reference to FIG. 2 is implemented by the input/output (I/O) 106. The input/output (I/O) 106 may be implemented by, for example, a keyboard and/or a touch panel.

The display 107 is configured by, for example, an organic EL display or a liquid crystal display. The display 107 may be used to implement the presentation unit 13 described with reference to FIG. 2.

Here, the program stored in the program storage area of the auxiliary memory 105 may be a program in which processing is performed in time series in accordance with the sequence of the prediction processing described herein, or may be a program in which processing is performed in parallel or at necessary timing such as when a call is executed.

Meanwhile, the program may be executed by a single computer, or may be executed in a distributed manner by a plurality of computers.

### [Hardware Configuration of Model Construction Apparatus]

Next, an example of a hardware configuration for implementing the model construction apparatus 2 with the above-described functions will be described with reference to the block diagram of FIG. 7.

As shown in FIG. 7, the model construction apparatus 2 can be implemented by a computer including, for example, a processor 202, a main memory 203, a communication interface (I/F) 204, an auxiliary memory 205, and an input/output (I/O) 206 connected via a bus 201, and by a program that controls these hardware resources. In addition, the model construction apparatus 2 is connected via the bus 201 to an external proteomics database 208; however, the apparatus need not be connected thereto if a means for transferring data is provided. The model construction apparatus 2 may also be connected via the bus 201 to a mass spectrometer, or need not be connected thereto if a means for transferring data is provided. Each hardware configuration included in the model construction apparatus 2 is substantially the same as that of the prediction apparatus 1 described with reference to FIG. 6.

The processor 202 and the main memory 203 cooperate to implement the respective functions of the model construction apparatus 2 including the model setting unit 20, the parameter setting unit 21, and the validation unit 22 shown in FIG. 3.

The memory 23 described with reference to FIG. 3 is implemented by the auxiliary memory 205. The auxiliary memory 105 includes a program storage area that stores the model construction program 23A to be executed by the model construction apparatus 2. The auxiliary memory 205 includes a storage area that stores a quantitative analysis program for performing quantitative analysis based on data measured with a mass spectrometer.

The proteomics database 208 includes a public proteomics database. For example, the proteomics database 208 may be implemented by a proteomics database such as The Human Protein Atlas (https://www.proteinatlas.org/about/download) and/or UniProt (https://www.uniprot.org/). As described above, the model construction apparatus 2 identifies expressed proteins by matching protein mass information obtained from a proteomics database against data measured with a mass spectrometer.

The model construction apparatus 2 may be connected to the mass spectrometer via a local area network (LAN) or another communication network, such that the measured data is automatically transmitted to the model construction apparatus 2. The mass spectrometer may be implemented by, for example, a liquid chromatography-mass spectrometry (LC-MS) system.

### [Operation of Model Construction Apparatus]

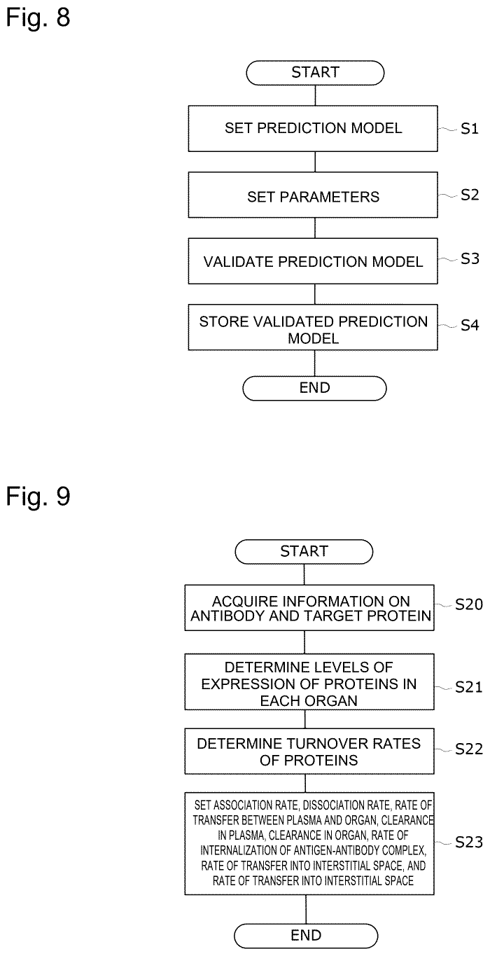

Next, the operation of the model construction apparatus 2 with the above configuration will be described in detail by using the flowchart of FIG. 8.

First, the model setting unit 20 sets a prediction model (step S1). Next, the parameter setting unit 21 sets parameters of the model set in step S1 (step S2). Then, the validation unit 22 validates the prediction model with the parameters set (step S3). After that, the validated prediction model is stored in the memory 23 (step S4). However, the validation unit 22 is optional, and step S3 is also an optional step.

Next, the operation of the parameter setting unit 21 of the model construction apparatus 2 in step S2 of FIG. 8 will be described with reference to the flowchart of FIG. 9. First, the kinetic parameter setting section 21A of the parameter setting unit 21 acquires information on the antibody and the target protein to be used for model construction, which model have been set by the model setting unit 20 in step S1 of FIG. 8 (step S20). Next, the protein amount determination section 21B determines the levels of expression of proteins in each organ on the basis of data obtained, for example, through proteomics analysis (step S21). Then, the turnover rate determination section 21C determines the turnover rates of the proteins in each organ on the basis of the rates of elimination of stable isotope-unlabeled proteins measured by TMT-SILAC (step S22).

After that, the kinetic parameter setting section 21A of the parameter setting unit 21 sets values for parameters incorporated into the prediction model on the basis of the levels of expression of the proteins in each organ as determined in step S21 and their turnover rates in each organ as determined in step S22 (step S23). In step S23, the parameter setting unit 21 sets the rate at which the antibody transfers between organ compartments. In addition, in step S23, the kinetic parameter setting section 21A sets the rate at which the antibody transfers from plasma to the interstitial space of each organ, and the rate at which the antibody transfers from the interstitial space of each organ to plasma. Further, in step S23, the kinetic parameter setting section 21A sets clearance of the antibody in plasma and clearance of the antibody in each organ.

In addition, in step S23, the kinetic parameter setting section 21A sets the rate at which an antigen-antibody complex, formed by binding of the antibody transferred from plasma to each of the plurality of organs to a target protein present in each corresponding organ, is internalized into cells of each corresponding organ. Further, in step S23, the kinetic parameter setting section 21A sets the rate at which the antibody transferred to each organ binds to the target protein in each corresponding organ. Furthermore, in step S23, the kinetic parameter setting section 21A sets the dissociation rate at which the antigen-antibody complex dissociates into the antibody and the target protein in plasma or in each of the plurality of organs. The parameters set are stored in the memory 23.

### [Operation of Prediction Apparatus]

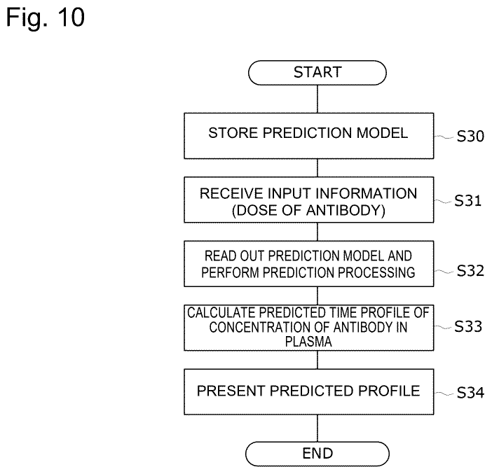

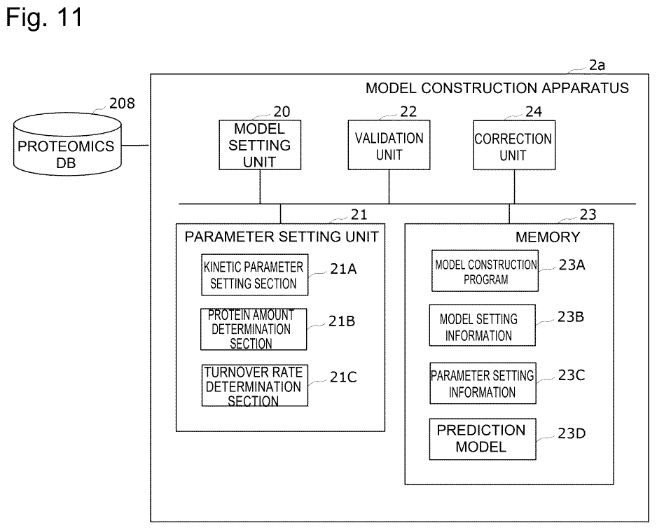

Next, the operation of the prediction apparatus 1 with the above configuration will be described by using the flowchart of FIG. 10.

First, the memory 10 stores a validated prediction model that has been constructed by the model construction apparatus 2 (step S30). Next, the input unit 11 receives input information about a dose of the prediction target antibody (step S31). The input unit 11 may receive input information through a user operation performed via a keyboard, a touch panel, or the like. Next, the prediction unit 12 reads out the prediction model stored in step S30, performs calculation of the prediction model on the basis of the prediction target antibody and its dose indicated by the input information, and outputs a predicted time profile of the concentration of the prediction target antibody in plasma (step S32).

In step S32, the time profile of the concentration of the antibody in plasma is predicted by solving the differential equations represented by Equations (a) to (r) and Formulas (1) to (7). After that, the presentation unit 13 presents the predicted profile output in step S32 (step S33).

The respective steps of the prediction method described with reference to FIGS. 8 to 10 are not necessarily required to be executed in this order, and some of the steps may be performed in a different order or in parallel.

As described above, the prediction system according to the first embodiment is used to construct a PBPK model that is constructed on the basis of pharmacokinetic data of an antibody and the model includes
a plurality of biological compartments and
as model parameters, inflow of the antibody into each compartment, outflow of the antibody from each compartment, and elimination of the antibody in each compartment, wherein the model parameters involving the elimination of the antibody in each compartment include an internalization rate at which an antigen-antibody complex, formed by binding between the antibody that has been transferred from plasma to each of a plurality of organs and an antigen protein possessed by each corresponding organ, is internalized into cells of each corresponding organ. It is thus possible to predict the time profile of the antibody concentration in blood on the basis of the prediction model that reflects in vivo kinetics while ensuring applicability.

According to the first embodiment described above, in predicting the antibody concentration based on a PBPK model composed of a plurality of biological compartments, the rate of internalization of a protein into cells in each organ is incorporated as a parameter. Accordingly, a prediction of the blood antibody concentration-time profile can be provided, which prediction takes into consideration a concentration range in which nonlinear elimination occurs due to a rapid decrease in the antibody concentration.

Meanwhile, according to the first embodiment described above, the prediction model is constructed in which the human body is divided into a plurality of organ compartments and a plasma compartment. It is thus possible to provide a physiologically more realistic prediction of the antibody concentration-time profile in humans.

In addition, according to the first embodiment described above, proteomics analysis is employed to identify the levels of expression of proteins in each organ and to determine the turnover rates of the proteins in each organ. Hence, the levels of expression of proteins and their kinetic parameters can be simultaneously measured across a plurality of organs, thereby enabling systematic inter-organ comparison and comprehensive analysis.

### Second Embodiment

Next, a prediction system according to the second embodiment will be described. In the first embodiment described above, a PBPK model configured to reproduce inter-organ pharmacokinetics in humans has been described. In contrast, in the second embodiment, a prediction model for humans is constructed based on a PBPK model configured to reproduce inter-organ pharmacokinetics in monkeys.

The configuration of the prediction system according to this embodiment is substantially the same as that of the prediction system according to the first embodiment described above. In the following description, elements identical to those of the first embodiment will not be described, and emphasis will be placed on elements that differ from the first embodiment.

The types and number of organ compartments constituting the prediction model constructed in this embodiment, as well as the connections therebetween, are the same as those of the model structure employed in the first embodiment. In addition, the types and number of parameters of the prediction model are substantially the same as those of the prediction model used in the first embodiment.

### [Functional Blocks of Model Construction Apparatus]

FIG. 11 is a block diagram showing the configuration of a model construction apparatus 2a included in the prediction system according to the second embodiment. The model construction apparatus 2a differs from the model construction apparatus 2 according to the first embodiment in that the model construction apparatus 2a further includes a correction unit 24.

As shown in FIG. 11, the model construction apparatus 2a includes a model setting unit 20, a parameter setting unit 21, a validation unit 22, a memory 23, and a correction unit 24.

The parameter setting unit 21 sets parameters of the prediction model for monkeys on the basis of metabolic data and omics data for monkeys. The parameter setting unit 21 determines the expression level of a protein, in each organ of cynomolgus monkeys and the rate of internalization of the target antigen into cells in each organ by using, for example, metabolic data of cynomolgus monkeys obtained through experiments and omics data of cynomolgus monkeys as acquired, for example, from an omics database of the model construction apparatus 2a. The parameter setting unit 21 sets values of the parameters of the prediction model by a procedure similar to that used in the case of the prediction model for humans.

The correction unit 24 uses scaling factors to adjust the predicted antibody concentration-time profile in monkeys after antibody administration on the basis of the target protein expression level in each organ and the rate of internalization into cells in each corresponding organ as set by the parameter setting unit 21, so that the predicted results match the observed plasma antibody concentration-time profile in monkeys when the antibody is administered. The scaling factors, which are each a correction coefficient by which the amount of protein contained in each monkey organ or the antigen internalization rate is multiplied, are determined, for example, by numerical calculation so as to fit the monkey model.

The correction unit 24 is used to obtain a prediction model for humans by multiplying, by each corresponding scaling factor that has been determined based on the prediction model for monkeys, the amount of antigen protein contained in each human organ and the internalization rate in the human prediction model. Although monkeys are used in this embodiment, other animals may be used, provided that they are anatomically similar to humans.

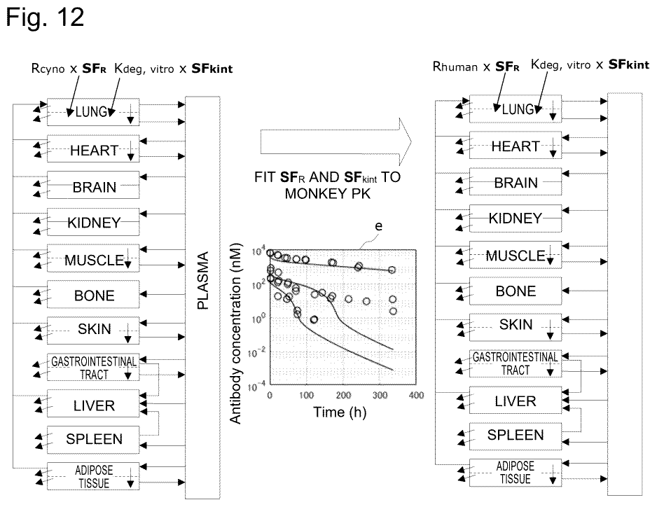

FIG. 12 is a diagram for explaining the scaling factors used by the correction unit 24. Region e in FIG. 12 illustrates the relationship between the experimentally obtained blood antibody concentration-time profile in cynomolgus monkeys after antibody administration and the antibody concentration-time profile values predicted by the prediction model in which parameters including the target protein expression level and internalization rate in each organ in cynomolgus monkeys as set by the parameter setting unit 21 are incorporated.

The correction unit 24 compares the experimental results shown in region e of FIG. 12 with predicted values generated by the prediction model, and determines two types of scaling factors, SF_{R} and SFᵢₙₜ, such that the predicted values fit the experimentally measured values. Quantitative protein measurements in each human organ and each monkey organ and protein metabolic rate values measured in vitro include various types of error.

Accordingly, the in vivo antibody concentration-time profile cannot be predicted accurately by directly using such measured values. By using two types of scaling factors, these errors can be corrected, thereby enabling more appropriate reproduction of the pharmacokinetics of the antibody in monkeys and humans.

In the prediction model for monkeys as shown on the left side of FIG. 12, the protein expression level in each monkey organ, R_{cyno}, is scaled to R_{cyno}×SF_{R}, namely a value obtained by multiplying R_{cyno} by the scaling factor SF_{R}, and the rate of internalization of the antigen into cells in each organ, K_{int, vitro}, is scaled to K_{int, vitro}×SFₖᵢₙₜ, namely a value obtained by multiplying K_{int, vitro} by the scaling factor SFₖᵢₙₜ.

Further, in the prediction model for humans as shown on the right side of FIG. 12, the target protein expression level in each human organ, Rₕᵤₘₐₙ, is scaled to Rₕᵤₘₐₙ×SF_{R}, namely a value obtained by multiplying Rₕᵤₘₐₙ by the scaling factor SF_{R}, and the rate of internalization of the antibody into cells in each organ, K_{int, vitro}, is scaled to K_{int, vitro}×SFₖᵢₙₜ, namely a value obtained by multiplying K_{int, vitro} by the scaling factor SFₖᵢₙₜ. In the human prediction model, as in the first embodiment, the protein amounts and expression levels in human organs, as well as the rates of antibody internalization into cells of the human organs, are used.

### [Operation of Model Construction Apparatus]

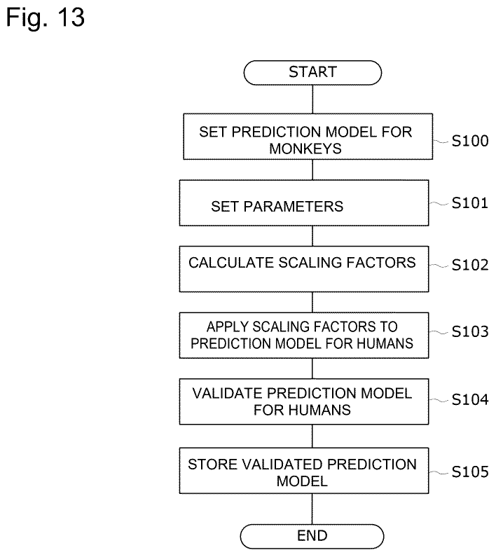

Next, the operation of the model construction apparatus 2 included in the prediction system with the above configuration will be described with reference to FIG. 13. First, the model setting unit 20 sets a prediction model for monkeys (step S100). In terms of the structure, the prediction model has the same organs and plasma compartment connections as those of the prediction model for humans as described with reference to FIG. 3. Next, the parameter setting unit 21 sets parameters of the prediction model (step S101). The parameter setting unit 21 sets values for parameters of the prediction model, including the protein expression level in each organ and the rate of antigen internalization into cells in each organ, as based on metabolic data and omics data derived from monkeys.

Next, the correction unit 24 performs fitting between the predicted values generated by the monkey prediction model set in step S101 and the antibody concentration-time profile obtained from a study on an antibody concentration-time profile after administration to monkeys, and determines scaling factors for the protein expression level in each and the rate of antibody internalization into cells in each corresponding organ (step S102).

The correction unit 24 corrects the monkey prediction model by applying the respective scaling factors to the protein expression level and the rate of antibody internalization in each organ. Further, the correction unit 24 applies the scaling factors determined in step S102 to the prediction model constructed for humans (step S103). In step S103, the human prediction model constructed based on protein data of human organs is corrected by applying the scaling factors determined in step S102 to the protein expression level in each organ and the rate of antibody internalization in each organ, respectively.

Subsequently, the validation unit 22 validates the human prediction model to which the scaling factors have been applied (step S104). After that, the validated human prediction model is stored in the memory 23 (step S105).

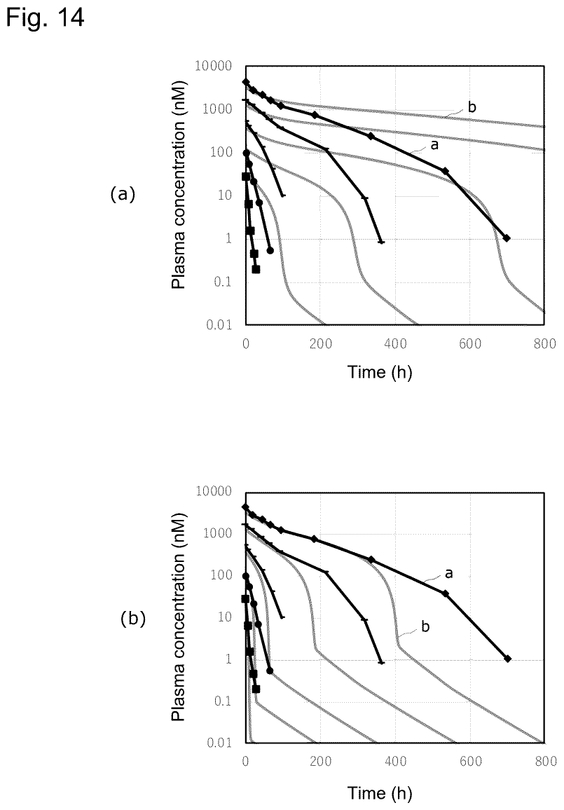

Here, effects of the prediction systems according to the first and second embodiments are described with reference to FIG. 14. FIGS. 14(a) and 14(b) illustrate antibody concentration-time profiles predicted by the prediction models for an antibody ibalizumab. The antigen against ibalizumab is CD4. FIG. 14(a) illustrates the plasma antibody concentration-time profile predicted by the human prediction model constructed by the prediction system according to the first embodiment. Curve a is plotted using the experimentally obtained antibody concentration-time profile values. Curve b is plotted using the predicted antibody concentration-time profile values obtained by the constructed prediction model. FIG. 14(b) illustrates the predicted values generated by a human prediction model to which the scaling factors, determined using the monkey prediction model constructed by the prediction system according to the second embodiment, have been applied.

The parameters of the prediction model were set as follows: clearance=11.97894 [mL/h]; binding affinity K_{D} between the antibody and the target protein=0.0822 [nM]; dissociation rate K_{off}=0.29592 [h⁻¹]; degradation rate Kᵢₙₜ=6.03712 [h⁻¹]; scaling factor SF_{R}=0.30276; and scaling factor SFₖᵢₙₜ=57.2364. For ibalizumab, in the prediction model of FIG. 14(b) to which the scaling factors were applied, the AAFE value was lower than that of the prediction model of FIG. 14(a), indicating improved prediction accuracy.

As described above, according to the prediction system of the second embodiment, the scaling factors determined based on the monkey prediction model are applied to the human prediction model, thereby capable of further improving the prediction accuracy of the plasma antibody concentration-time profile. In particular, since actual experimental data can be obtained in monkeys to correct omics-derived protein expression levels and internalization rates, the prediction accuracy can be further improved.

As described above, in the first and second embodiments, the prediction apparatus 1 is not limited to a case where the prediction model constructed by the model construction apparatus 2 is acquired and prediction processing is performed locally. For example, a system configuration may be employed in which the prediction processing is performed in a cloud environment. In this case, the model construction apparatus 2 is configured in a cloud server, and the prediction apparatus 1 transmits input information to the cloud server. The cloud server performs the prediction processing and returns the predicted values to the prediction apparatus 1.

In addition, in the first and second embodiments described above, the case has been described in which the protein amount determination section 21B uses proteomics data to identify protein expression levels in each organ. However, the identification of the protein expression levels in each organ is not limited thereto. For example, instead of using proteomics data, mRNA expression levels may be used to estimate the protein amounts in each organ, and the estimated amounts may be employed as the protein expression levels. For example, RNA sequencing may be used to obtain mRNA expression levels. In this case, data on the mRNA expression levels is used to construct a monkey prediction model and calculate the scaling factors. Further, the data on the mRNA expression levels and the scaling factors may be used to estimate protein expression levels in each organ applicable to humans.

Meanwhile, in the first and second embodiments described above, the prediction system has been described as including the prediction apparatus 1 and the model construction apparatus 2. Nevertheless, the prediction system may be configured to consist of the model construction apparatus 2.

In addition, in the first and second embodiments described above, the case has been described in which the prediction system employs a prediction model composed of a plurality of organ compartments and a plasma compartment. However, the compartments constituting the prediction model may further include a lymph compartment.

Meanwhile, in the first and second embodiments described above, the case has been described in which the prediction system constructs a prediction model based on the differential equations set forth in Equations (a) to (r) and Formulas (1) to (7). However, the prediction model is not limited to being expressed in the form of differential equations. For example, the prediction model may be constructed using machine learning techniques including neural networks, Gaussian processes, random forests, and probabilistic models, or using algorithmic approaches such as algebraic equations or rule-based methods.

In addition, the prediction program according to the first and second embodiment described above may be recorded on a computer-readable recording medium, such as a hard disk or a flexible disk, and executed by a computer upon being read from the recording medium. The program may also be provided in the form of a transmission medium distributable via a network such as the Internet.

Note that the various functional blocks, modules, and circuits described in connection with the embodiments disclosed herein may be implemented by a general-purpose processor, a GPU, a digital signal processor (DSP), an application-specific integrated circuit (ASIC), an FPGA or other programmable logic device, discrete gate or transistor logic devices, discrete hardware components, or any combination thereof designed to realize the functions described above.

A microprocessor may be used as the general-purpose processor; alternatively, a processor, controller, microcontroller, or state machine in the art may be used. The processor may be implemented, for example, as a combination of a DSP and a microprocessor, multiple microprocessors, one or more microprocessors connected to a DSP core, or as any combination of such optional component computing devices.

While certain embodiments of the prediction system, prediction method, and prediction program according to the present disclosure have been described above, the present disclosure is not limited to the described embodiments, and various conceivable modifications may be made by those skilled in the art within the scope of the disclosure as defined in the CLAIMS.

The functionality of the elements disclosed herein may be implemented using circuitry or processing circuitry which includes general purpose processors, special purpose processors, integrated circuits, ASICs (“Application Specific Integrated Circuits”), FPGAs (“Field Programmable Gate Arrays”), conventional circuitry and/or combinations thereof which are programmed, using one or more programs stored in one or more memories, or otherwise configured to perform the disclosed functionality. Processors are considered processing circuitry or circuitry as they include transistors and other circuitry therein. The processor may be a programmed processor which executes a program stored in a memory. In the disclosure, the circuitry, units, means, and sections are hardware that carry out or are programmed to perform the recited functionality. The hardware may be any hardware disclosed herein which is programmed or configured to carry out the recited functionality.

As used herein, a “memory” or “memory module” may refer to a non-transitory computer-readable medium. The non-transitory computer-readable medium may store instructions. For example, the non-transitory computer-readable medium may store a set of instructions which, when executed by one or more processors, cause the circuitry described above to perform operations described herein, including without limitation: constructing a physiologically based pharmacokinetic (PBPK) model; storing the PBPK model in memory; receiving input information including a candidate dose of a prediction target antibody; calculating, using the PBPK model and the candidate dose, a predicted time profile of a plasma concentration of the prediction target antibody; determining, based on the predicted time profile, a dose of the prediction target antibody for administration to a subject; and outputting the determined dose.

### REFERENCE SIGNS LIST

1 . . . Prediction apparatus, 2 . . . Model construction apparatus, 10, 23 . . . Memory, 11 . . . Input unit, 12 . . . Prediction unit, 13 . . . Presentation unit, 20 . . . Model setting unit, 21 . . . Parameter setting unit, 22 . . . Validation unit, 101, 201 . . . Bus, 102, 202 . . . Processor, 103, 203 . . . Main memory, 104, 204 . . . Communication I/F, 105, 205 . . . Auxiliary memory, 106, 206 . . . Input/Output I/O, 107, 207 . . . Display, 208 . . . Proteomics DB, NW . . . Network.

1. A system comprising:
circuitry configured to:
store, in a memory, a compartmental model including a plurality of compartment nodes and, as model parameters, first flow rates representing entry of a first substance into each compartment node, second flow rates representing exit of the first substance from each compartment node, and elimination parameters representing removal of the first substance in each compartment node, the elimination parameters including an internalization rate at which a complex, formed by binding between the first substance that has been transferred from a source compartment node to a target compartment node and a second substance associated with the target compartment node, is removed from the target compartment node, and the compartmental model being constructed based on reference interaction data;
receive, via a communication interface, input data indicating a dose of the first substance;
numerically integrate, using the compartmental model, a plurality of coupled differential equations to generate a predicted time profile of a concentration of the first substance in a reference compartment node; and
output the predicted time profile to a display device.

2. The system according to claim 1, wherein
the first substance is an antibody, the second substance is an antigen protein possessed by an organ, the complex is an antigen-antibody complex that is internalized into cells of the organ, and the reference interaction data comprises pharmacokinetic data of the antibody.

3. The system according to claim 2, wherein
the antibody is an IgG antibody of a subclass selected from the group consisting of IgG1, IgG2, IgG3, and IgG4.

4. The system according to claim 1, wherein the compartmental model and the model parameters of the compartmental model are set by the circuitry prior to being stored in the memory.

5. The system according to claim 1, wherein
the plurality of compartment nodes comprise a plasma compartment and a plurality of organ compartments, the plurality of organ compartments comprising a lung compartment and a plurality of non-lung organ compartments, and
the reference compartment node is the plasma compartment.

6. The system according to claim 5, wherein
the compartmental model is a physiologically based pharmacokinetic (PBPK) model of a human, and the plurality of non-lung organ compartments comprise a heart compartment, a brain compartment, a kidney compartment, a muscle compartment, a bone compartment, a skin compartment, a gastrointestinal tract compartment, a liver compartment, a spleen compartment, and an adipose tissue compartment.

7. The system according to claim 5, wherein
the compartmental model assumes that the first substance is administered into the plasma compartment, and
the model parameters include a transfer amount of the first substance between the plasma compartment and each of the plurality of non-lung organ compartments, a transfer amount of the first substance between the plurality of non-lung organ compartments and the lung compartment, and a transfer amount of the first substance between the lung compartment and the plasma compartment.

8. The system according to claim 7, wherein
each transfer amount is calculated based on a transport flow rate of the first substance.

9. The system according to claim 7, wherein the plurality of organ compartments comprise:
a first organ compartment in which a change over time in an amount of the first substance within the first organ compartment is calculated by subtracting, from an inflow amount from the plasma compartment, an outflow amount to the lung compartment and an elimination amount due to a clearance;
a second organ compartment in which a change over time in an amount of the first substance in a vascular region of the second organ compartment is calculated by subtracting, from an inflow amount from the plasma compartment, an outflow amount to the lung compartment, a transfer amount to an interstitial region of the second organ compartment, and an elimination amount due to a clearance, a change over time in an amount of the first substance in the interstitial region is calculated by subtracting, from the transfer amount, an amount of binding to the second substance and an amount transferred via a lymphatic flow to the plasma compartment, and adding an amount of dissociation from the second substance, and a change over time in an amount of the complex is calculated by subtracting, from an amount of the first substance bound to the second substance, the amount of dissociation and a product of the amount of the complex and the internalization rate; and
a third organ compartment in which a change over time in an amount of the first substance is calculated by subtracting, from an inflow amount from the plasma compartment, an outflow amount to the lung compartment, an elimination amount due to a clearance, and an amount of binding to the second substance, and adding an amount of dissociation from the second substance.

10. The system according to claim 5, wherein
an amount of the first substance transferred from an interstitial region of one of the plurality of organ compartments to the plasma compartment is determined based on a concentration of the first substance in the interstitial region and a lymph flow rate of the one of the plurality of organ compartments.

11. The system according to claim 5, wherein:
the input data further indicates a designated organ compartment of the plurality of organ compartments;
the predicted time profile generated by the circuitry comprises a predicted time profile of a concentration of the first substance in the designated organ compartment; and
the predicted time profile of the concentration of the first substance in the designated organ compartment is output to the display device.

12. The system according to claim 2, wherein
an amount of binding between the antibody and the antigen protein in each of a plurality of organs is determined based on a concentration of the antibody in an interstitial region of the organ, a concentration of the antigen protein that is expressed on a cell membrane and is unbound to the antibody, and an association rate,
an amount of the antibody dissociating from the antigen protein in each of the plurality of organs is determined based on a concentration of the antigen-antibody complex and a dissociation rate, and
an amount of the antigen-antibody complex that is internalized into cells of each of the plurality of organs is determined based on the amount of the antigen-antibody complex and the internalization rate.

13. The system according to claim 2, wherein
an amount of the antigen protein possessed by each of a plurality of organs is determined by a product of a total protein amount per unit weight of the organ, a proportion of the antigen protein relative to the total protein amount, and a weight of the organ.

14. The system according to claim 2, wherein
an amount of the antigen protein possessed by each of a plurality of organs is determined by a proteomics analysis.

15. The system according to claim 14, wherein
the proteomics analysis comprises digesting proteins derived from each of the plurality of organs with a protease, separating resulting peptides by liquid chromatography, and analyzing the peptides using a mass spectrometer.

16. The system according to claim 2, wherein
the amount of the antigen protein possessed by each of a plurality of organs and the internalization rate are determined by multiplying, by a scaling factor, a corresponding value obtained from a non-human animal, the scaling factor being determined by numerical calculation so as to fit a time profile of a concentration of the antibody in the non-human animal.

17. A method comprising:
storing, in a memory, a compartmental model including a plurality of compartment nodes and, as model parameters, first flow rates representing entry of a first substance into each compartment node, second flow rates representing exit of the first substance from each compartment node, and elimination parameters representing removal of the first substance in each compartment node, the elimination parameters including an internalization rate at which a complex, formed by binding between the first substance that has been transferred from a source compartment node to a target compartment node and a second substance associated with the target compartment node, is removed from the target compartment node, and the compartmental model being constructed based on reference interaction data;
receiving, via a communication interface, input data indicating a dose of the first substance;
numerically integrating, using the compartmental model, a plurality of coupled differential equations to generate a predicted time profile of a concentration of the first substance in a reference compartment node; and
outputting the predicted time profile to a display device.

18. The method according to claim 17, wherein
the first substance is an antibody, the second substance is an antigen protein possessed by an organ, the complex is an antigen-antibody complex that is internalized into cells of the organ, and the reference interaction data comprises pharmacokinetic data of the antibody.

19. A non-transitory computer-readable medium storing instructions that, when executed by one or more processors, cause the one or more processors to:
store, in a memory, a compartmental model including a plurality of compartment nodes and, as model parameters, first flow rates representing entry of a first substance into each compartment node, second flow rates representing exit of the first substance from each compartment node, and elimination parameters representing removal of the first substance in each compartment node, the elimination parameters including an internalization rate at which a complex, formed by binding between the first substance that has been transferred from a source compartment node to a target compartment node and a second substance associated with the target compartment node, is removed from the target compartment node, and the compartmental model being constructed based on reference interaction data;
receive, via a communication interface, input data indicating a dose of the first substance;
numerically integrate, using the compartmental model, a plurality of coupled differential equations to generate a predicted time profile of a concentration of the first substance in a reference compartment node; and
output the predicted time profile to a display device.

20. The non-transitory computer-readable medium according to claim 19, wherein
the first substance is an antibody, the second substance is an antigen protein possessed by an organ, the complex is an antigen-antibody complex that is internalized into cells of the organ, and the reference interaction data comprises pharmacokinetic data of the antibody.
